(19) United States
(12) Patent Application Publication
(10) Pub. No.: US 2025/0281464 A1
(43) Pub. Date: Sep. 11, 2025

(54) TREATMENT OF PATHOGENIC NEISSERIA SP. INFECTION WITH TRIAZOLE ANTIFUNGAL AGENTS

(71) Applicant: Virginia Tech Intellectual Properties, Inc., Blacksburg, VA (US)

(72) Inventor: Mohamed SELEEM, Blacksburg, VA (US)

(21) Appl. No.: 18/861,456

(22) PCT Filed: Apr. 28, 2023
(86) PCT No.: PCT/US2023/020348

§ 371 Date: Oct. 29, 2024

Related U.S. Application Data
(60) Provisional application No. 63/336,513, filed on Apr. 29, 2022.

(51) Int. Cl. A61K 31/427; A61K 31/546; A61K 31/7052; A61P 31/04

(52) U.S. Cl. CPC A61K 31/427; A61K 31/546; A61K 31/7052; A61P 31/04

(57) ABSTRACT

Described in several example embodiments herein are compositions and methods to treat and/or prevent infection with a Neisseria species (sp) infection and/or a disease or disorder caused by a Neisseria species (sp) or a symptom thereof. In some embodiments, the Neisseria sp. is Neisseria gonorrhoeae or N. meningitidis.

### CROSS-REFERENCE TO RELATED APPLICATIONS

This application claims the benefit of and priority to U.S. Provisional Patent Application No. 63/336,513, filed on Apr. 29, 2022, entitled “TREATMENT OF N. GONORRHOEA INFECTION WITH RAVUCONAZOLE,” the contents of which is incorporated by reference herein in its entirety.

### TECHNICAL FIELD

The subject matter disclosed herein is generally directed to compositions of methods for treating Neisseria infections (e.g., Neisseria gonorrhoeae (N. gonorrhoeae) and/or Neisseria meningitidis) infection in a subject in need thereof.

### BACKGROUND

Neisseria species are generally well-adapted to mammalian hosts. The closely related N. gonorrhoeae and N. meningitidis are the only two species of the genus recognized as strict human pathogens. N. gonorrhoeae represents an urgent public health threat due to the rapid emergence of resistance to current antibiotics and the limited number of anti-gonococcal agents currently in clinical trials. Neisseria gonorrhoeae is the second most reported notifiable disease in the United States and one of the most important antimicrobial resistance threats worldwide. The CDC has listed drug-resistant N. gonorrhoeae at the highest possible threat level to public health. With no effective vaccines available, the development of effective antibacterial agents is a critical priority.

N. meningitidis can be pathogenic and cause cerebrospinal meningitis and septicemia, with significant mortality and morbidity worldwide. Although currently rare N. meningitidis can develop resistance to antibiotics. As such alternate approach's to treatment is warranted

Citation or identification of any document in this application is not an admission that such a document is available as prior art to the present invention.

### SUMMARY

Described in certain example embodiments herein are methods of treating a Neisseria species (sp) infection or a symptom thereof in a subject in need thereof, the method comprising administering, to the subject in need thereof, an amount of a triazole antifungal agent or a derivative thereof, or a prodrug thereof.

In some embodiments the Neisseria sp. Is Neisseria gonorrhoeae or N. meningitidis.

Described in certain example embodiments herein are compositions and formulations thereof effective to treat and/or prevent Neisseria gonorrhoeae infection and/or symptom(s) thereof in a subject in need thereof. In certain example embodiments, the composition or formulation thereof comprises an effective amount of ravuconazole or a derivative thereof.

Described in certain example embodiments herein are pharmaceutical formulations comprising an amount of ravuconazole or a derivative thereof and pharmaceutically acceptable carrier. In certain example embodiments, the pharmaceutical formulation is effective to treat or prevent Neisseria gonorrhoeae infection and/or symptom(s) thereof in a subject in need thereof.

Described in certain example embodiments herein and methods of treating and/or preventing Neisseria gonorrhoeae infection and/or symptom(s) thereof in a subject in need thereof. In certain example embodiments, the method of treating and/or preventing Neisseria gonorrhoeae infection and/or symptom(s) thereof in a subject in need thereof comprises administering an amount, such as an effective amount, of ravuconazole or a derivative thereof or a pharmaceutical formulation thereof to the subject in need thereof.

Described in certain example embodiments herein are methods of treating a Neisseria gonorrhoeae infection or a symptom thereof in a subject in need thereof, the method comprising administering, to the subject in need thereof, an amount of a triazole antifungal agent or a derivative thereof, or a prodrug thereof.

In certain example embodiments, the triazole antifungal agent is ravuconazole, isavuconazole, itraconazole, terconazole, ketoconazole, or any combination thereof.

In certain example embodiments, the amount of ravuconazole ranges from about 20 mg/kg to about 60 mg/kg.

In certain example embodiments, the amount of ravuconazole is about 40 mg/kg.

In certain example embodiments, the amount of the triazole antifungal agent ranges from about 10 mg to 1000 mg. or more.

In certain example embodiments, the amount of the triazole antifungal agent is administered in a single dose.

In certain example embodiments, the single dose is a single, one-time dose.

In certain example embodiments, the amount of the triazole antifungal agent is administered in two or more doses.

In certain example embodiments, the antifungal agent does not disrupt a microbiome in a subject in need thereof.

In certain example embodiments, administering an amount of a triazole antifungal agent comprises oral administration.

In certain example embodiments, the method further comprises administering an amount of an antibiotic.

In certain example embodiments, the antibiotic comprises azithromycin, penicillin, ciprofloxacin, ceftriaxone, cefixime, tetracycline, or any combination thereof.

In certain example embodiments, the amount of the antibiotic is about 0.001 LM to Ig or more.

In certain example embodiments, the Neisseria gonorrhoeae is an antibiotic resistant Neisseria gonorrhoeae. In certain example embodiments, the Neisseria gonorrhoeae is resistant to azithromycin, penicillin, ciprofloxacin, ceftriaxone, cefixime, tetracycline, or any combination thereof.

Described in certain example embodiments herein are pharmaceutical formulations comprising an amount of a triazole antifungal agent or a derivative thereof, or a prodrug thereof effective to treat Neisseria gonorrhoeae infection in a subject in need thereof; and a pharmaceutically acceptable carrier.

In certain example embodiments, the triazole antifungal agent is ravuconazole, isavuconazole, itraconazole, terconazole, ketoconazole, or any combination thereof.

In certain example embodiments, the amount of the amount of the triazole antifungal agent ranges from about 10 mg to 1000 mg or more.

In certain example embodiments, the pharmaceutical formulation comprises an amount of an antibiotic.

In certain example embodiments, the antibiotic is azithromycin, penicillin, ciprofloxacin, ceftriaxone, cefixime, tetracycline, or any combination thereof.

In certain example embodiments, the amount of the antibiotic is about 0.001 μM to 1 g or more.

In certain example embodiments, the pharmaceutical formulation is adapted for a single one time administration.

In certain example embodiments, the pharmaceutical formulation is adapted for oral administration or topical administration, optionally via administration to the vagina.

Described in certain example embodiments herein are kits comprising a pharmaceutical formulation as described herein; and instructions in a tangible medium of expression directing administration to a subject in need thereof, wherein the subject in need thereof has a Neisseria gonorrhoeae infection.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Efficacy of Ravuconazole one oral dose in a mouse model against N. gonorrhoeae FA1090. N. gonorrhoeae burden (average±standard deviation log 10 CFU/mL) after treatment with a single dose of ravuconazole (40 mg/kg) (fosravuconazole/E-1224 Prodrug) via oral gavage. As a negative control, mice (n=8) were administered the vehicle (water), As a positive control, a group of mice received a single intraperitoneal dose of ceftriaxone (15 mg/kg in water). Vaginal swabs were collected daily and cultured to determine the number of viable bacteria. The limit of detection was 10 CFU/mL. The data were analyzed via a two-way ANOVA followed by Dunnett's test for multiple comparisons. Asterisks indicate a statistical significant difference between mice treated with either ravuconazole or ceftriaxone as compared to the vehicle-treated mice.

FIG. 2A-2C—Efficacy of Ravuconazole one oral dose in a mouse model against drug-resistant strains. N. gonorrhoeae (FA1090) (FIG. 2A), Azithromycin resistant N. gonorrhoeae CDC-181 (FIG. 2B), and ceftriaxone-resistant N. gonorrhoeae WHO-X (FIG. 2C). N. gonorrhoeae burden (average±standard deviation log 10 CFU/mL) after treatment with a single dose of ravuconazole (160 mg/kg) (fosravuconazole/E-1224 Prodrug) via oral gavage. As a negative control, mice (n=6) were administered the vehicle (water), As a positive control, a group of mice received a single intraperitoneal dose of ceftriaxone (15 mg/kg in water). Vaginal swabs were collected daily and cultured to determine the number of viable bacteria. The limit of detection was 10 CFU/mL. The data were analyzed via a two-way ANOVA followed by Dunnett's test for multiple comparisons. Asterisks (*) indicate a statistically significant difference from control. A pound (#) indicates a statistically significant difference from ceftriaxone (P<0.05).

FIG. 3—Efficacy of 3 doses of isavuconazole against the vaginal burden of N. gonorrhoeae. N. gonorrhoeae burden (average±standard deviation log₁₀ CFU/mL) after treatment with isavuconazole (100 mg/kg and 160 mg/kg) orally for 3 consecutive days. N=6. The data were analyzed via a two-way ANOVA followed by Dunnett's test for multiple comparisons. Asterisks (*) indicate a statistically significant difference from the control (P<0.05). As a negative control, mice (n=6) were administered the vehicle (water), As a positive control, a group of mice received a single intraperitoneal dose of ceftriaxone (15 mg/kg in water).

FIG. 4—Time-kill assay of azoles, DMSO, and control antibiotics at 10×MIC against N. gonorrhoeae FA1090. Each agent was tested in triplicate. Error bars represent standard deviation values and is too small to be visualized for some timepoints.

FIG. 5—Intracellular clearance assay. The results shown are an average of 2 independent experiments with the standard deviation indicated. Each experiment consisted of 6 wells per treatment. Cells treated with azoles and azithromycin were compared to cells treated with the negative control (DMSO) (*) and cells treated with ceftriaxone (#) (P<0.01, analyzed via an unpaired t-test).

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

The term “biocompatible”, as used herein, refers to a substance or object that performs its desired function when introduced into an organism without inducing significant inflammatory response, immunogenicity, or cytotoxicity to native cells, tissues, or organs, or to cells, tissues, or organs introduced with the substance or object. For example, a biocompatible product is a product that performs its desired function when introduced into an organism without inducing significant inflammatory response, immunogenicity, or cytotoxicity to native cells, tissues, or organs.

Biocompatibility, as used herein, can be quantified using the following in vivo biocompatibility assay. A material or product is considered biocompatible if it produces, in a test of biocompatibility related to immune system reaction, less than 50%, 45%, 40%, 35%, 30%, 25%, 20%, 15%, 10%, 8%, 6%, 5%, 4%, 3%, 2%, or 1% of the reaction, in the same test of biocompatibility, produced by a material or product the same as the test material or product except for a lack of the surface modification on the test material or product. Examples of useful biocompatibility tests include measuring and assessing cytotoxicity in cell culture, inflammatory response after implantation (such as by fluorescence detection of cathepsin activity), and immune system cells recruited to implant (for example, macrophages and neutrophils).

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

As used herein, “derivative” can refer to any compound having the same or a similar core structure to the compound but having at least one structural difference, including substituting, deleting, and/or adding one or more atoms or functional groups. The term “derivative” does not mean that the derivative is synthesized from the parent compound either as a starting material or intermediate, although this may be the case. The term “derivative” can include prodrugs, or metabolites of the parent compound. Derivatives include compounds in which free amino groups in the parent compound have been derivatized to form amine hydrochlorides, p-toluene sulfoamides, benzoxycarboamides, t-butyloxycarboamides, thiourethane-type derivatives, trifluoroacetylamides, chloroacetylamides, or formamides. Derivatives include compounds in which carboxyl groups in the parent compound have been derivatized to form methyl and ethyl esters, or other types of esters or hydrazides. Derivatives include compounds in which hydroxyl groups in the parent compound have been derivatized to form O-acyl or O-alkyl derivatives. Derivatives include compounds in which a hydrogen bond donating group in the parent compound is replaced with another hydrogen bond donating group such as OH, NH, or SH. Derivatives include replacing a hydrogen bond acceptor group in the parent compound with another hydrogen bond acceptor group such as esters, ethers, ketones, carbonates, tertiary amines, imine, thiones, sulfones, tertiary amides, and sulfides. “Derivatives” also includes extensions of the replacement of the cyclopentane ring with saturated or unsaturated cyclohexane or other more complex, e.g., nitrogen-containing rings, and extensions of these rings with side various groups.

As used herein, “infection” as used herein refers to presence of an infective agent, such as a pathogen, e.g., a microorganism, in or on a subject, which, if its presence or growth were inhibited, would result in a benefit to the subject. Hence, the term refers to the state produced by the establishment, more particularly invasion and multiplication, of an infective agent, such as a pathogen, e.g., a microorganism, in or on a suitable host. An infection may produce tissue injury and progress to overt disease through a variety of cellular and toxic mechanisms.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “positive control” refers to a “control” that is designed to produce the desired result, provided that all reagents are functioning properly and that the experiment is properly conducted.

As used herein, “substituted,” refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, and polypeptide groups. Such alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, and polypeptide groups can be further substituted. “Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, and polypeptide groups. Such alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀cyclic, substituted C₃-C₂₀ cyclic, heterocyclic, substituted heterocyclic, amino acid, poly(lactic-co-glycolic acid), peptide, and polypeptide groups can be further substituted.

A “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein, “synergistic effect,” “synergism,” or “synergy” can refer to an effect arising between two or more molecules, compounds, substances, factors, or compositions that that is greater than or different from the sum of their individual effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used interchangeably herein, the terms “sufficient” and “effective,” can refer to an amount (e.g., mass, volume, dosage, concentration, and/or time period) needed to achieve one or more desired result(s). For example, a therapeutically effective amount refers to an amount needed to achieve one or more therapeutic effects.

As used herein, “therapeutic” refers to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as gonorrhea and/or infection with N. gonorrhoeae. The effect can be therapeutic in terms of a partial or complete cure of a disease, condition, symptom or adverse effect attributed to the disease, disorder, or condition. The term “treatment” as used herein covers any treatment of gonorrhea and/or infection with N. gonorrhoeae, in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

All publications, published patent documents, and patent applications cited herein are hereby incorporated by reference to the same extent as though each individual publication, published patent document, or patent application was specifically and individually indicated as being incorporated by reference.

### OVERVIEW

N. gonorrhoeae represents an urgent public health threat due to the rapid emergence of resistance to current antibiotics and the limited number of anti-gonococcal agents currently in clinical trials (1). Neisseria gonorrhoeae is the second most reported notifiable disease in the United States and one of the most important antimicrobial resistance threats worldwide (2). The CDC has listed drug-resistant N. gonorrhoeae at the highest possible threat level to public health. With no effective vaccines available, the development of effective antibacterial agents is a critical priority.

Few effective therapeutic options are available to treat gonorrhea. Current guidelines recommend treating gonorrhea with injectable ceftriaxone. However, increasing resistance to this treatment option has been reported, which has resulted in multidrug-resistant N. gonorrhoeae (i.e., super gonorrhea) becoming a critical public health concern. Compounding the problem further is that over the last 3 decades, almost no new classes of antibiotic drugs have been approved, raising a serious concern that super gonorrhea infections may be untreatable in the near future. (1-7).

Drug development is a time-consuming, costly, and high-risk venture given few compounds successfully make it through stringent regulatory requirements to the marketplace. Collectively, this points to a critical need for the identification of novel strategies to develop antibiotics to deal with this challenging health issue.

With that said, embodiments disclosed herein can provide compositions effective to treat and/or prevent Neisseria gonorrhoeae infection and/or symptom(s) thereof in a subject in need thereof. In certain example embodiments, the composition comprises an effective amount of ravuconazole or a derivative thereof. Also described in certain example embodiments herein are pharmaceutical formulations comprising an amount of ravuconazole or a derivative thereof and pharmaceutically acceptable carrier. In certain example embodiments, the pharmaceutical formulation is effective to treat or prevent Neisseria gonorrhoeae infection and/or symptom(s) thereof in a subject in need thereof. Also described in certain example embodiments herein and methods of treating and/or preventing Neisseria gonorrhoeae infection and/or symptom(s) thereof in a subject in need thereof. In certain example embodiments, the method of treating and/or preventing Neisseria gonorrhoeae infection and/or symptom(s) thereof in a subject in need thereof comprises administering an amount, such as an effective amount, of ravuconazole or a derivative thereof or a pharmaceutical formulation thereof to the subject in need thereof.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is u'eful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavemosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

In certain example embodiments, the pharmaceutical formulation contains an amount of a triazole antifungal agent or a derivative thereof, or a prodrug thereof effective to treat Neisseria gonorrhoeae infection in a subject in need thereof, and a pharmaceutically acceptable carrier. In some embodiments, the derivative of the triazole antifungal agent is a substituted derivative.

In certain example embodiments, the triazole antifungal agent or a derivative thereof or a prodrug thereof, is ravuconazole or a derivative thereof or a prodrug thereof, isavuconazole or a derivative thereof or a prodrug thereof, itraconazole or a derivative thereof or a prodrug thereof, ketoconazole or a derivative thereof or a prodrug thereof, terconazole or a derivative thereof or a prodrug thereof, or any combination thereof. In some embodiments, the antifungal agent is ravuconazole or a derivative thereof or a prodrug thereof. In some embodiments, the antifungal agent is isavuconazole or a derivative thereof or a prodrug thereof. In some embodiments, the antifungal agent is itraconazole or a derivative thereof or a prodrug thereof. In some embodiments, the antifungal agent is ketoconazole or a derivative thereof or a prodrug thereof. In some embodiments, the antifungal agent is terconazole or a derivative thereof or a prodrug thereof. In some embodiments, the antifungal agents include ravuconazole or a derivative thereof or a prodrug thereof and isavuconazole or a derivative thereof or a prodrug thereof. In some embodiments, the antifungal agents include ravuconazole or a derivative thereof or a prodrug thereof and itraconazole or a derivative thereof or a prodrug thereof. In some embodiments, the antifungal agents include isavuconazole or a derivative thereof or a prodrug thereof and itraconazole or a derivative thereof or a prodrug thereof. In some embodiments, the antifungal agents include ravuconazole or a derivative thereof or a prodrug thereof, isavuconazole or a derivative thereof or a prodrug thereof, and itraconazole or a derivative thereof or a prodrug thereof.

In certain example embodiments, the amount of the amount of each of the triazole antifungal agent or a derivative thereof or a prodrug thereof independently ranges from about 10 mg to 1000 mg or more. In certain example embodiments, the amount of traizole antifungal agent or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of ravuconazole or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of isavuconazole or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of itraconazole or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of ketoconazole or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of terconazole or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of ravucanazole or a derivative thereof or a prodrug thereof ranges from about 10 mg to 500 mg. In some embodiments, the amount of ravuconazole or a derivative thereof or a prodrug thereof is any non-zero number ranging from about 0 to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 mg. In some embodiments, the amount of ravucanazole or a derivative thereof or a prodrug thereof ranges from about 50 mg to 300 mg.

In some embodiments, the amount of itraconazole or a derivative thereof or a prodrug thereof ranges from about 10 mg to 500 mg. In some embodiments, the amount of itraconazole or a derivative thereof or a prodrug thereof is any non-zero number ranging from about 0 to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 mg. In some embodiments, the amount of itraconazole or a derivative thereof or a prodrug thereof ranges from about 50 mg to 300 mg.

In some embodiments, the amount of isavuconazole or a derivative thereof or a prodrug thereof ranges from about 10 mg to 500 mg. In some embodiments, the amount of isavuconazole or a derivative thereof or a prodrug thereof is any non-zero number ranging from about 0 to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 mg. In some embodiments, the amount of isavuconazole or a derivative thereof or a prodrug thereof ranges from about 50 mg to 300 mg.

In some embodiments, the amount of ketoconazole or a derivative thereof or a prodrug thereof ranges from about 10 mg to 500 mg. In some embodiments, the amount of ketoconazole or a derivative thereof or a prodrug thereof is any non-zero number ranging from about 0 to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 mg. In some embodiments, the amount of ketoconazole or a derivative thereof or a prodrug thereof ranges from about 50 mg to 300 mg.

In some embodiments, the amount of terconazole or a derivative thereof or a prodrug thereof ranges from about 10 mg to 500 mg. In some embodiments, the amount of terconazole or a derivative thereof or a prodrug thereof is any non-zero number ranging from about 0 to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 mg. In some embodiments, the amount of terconazole or a derivative thereof or a prodrug thereof ranges from about 50 mg to 300 mg.

In certain example embodiments, the pharmaceutical formulation comprises an amount of an antibiotic or a derivative thereof or a prodrug thereof. In some embodiments, the antibiotic is one effective to treat N. gonorrhoeae infection. In certain example embodiments, the antibiotic is azithromycin or a derivative thereof or a prodrug thereof, penicillin or a derivative thereof or a prodrug thereof, ciprofloxacin or a derivative thereof or a prodrug thereof, ceftriaxone or a derivative thereof or a prodrug thereof, cefixime or a derivative thereof or a prodrug thereof, tetracycline or a derivative thereof or a prodrug thereof, or any combination thereof.

In certain example embodiments, the amount of the antibiotic is about 0.001 μM to 1 g or more. In some embodiments, the amount of antibiotic or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of azithromycin or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of penicillin or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of ciprofloxacin or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of ceftriaxone or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of cefixime or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of tetracycline or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, only one dose of the pharmaceutical formulation is effective to treat the N. gonorrhoeae infection. As such in certain example embodiments, the pharmaceutical formulation is adapted for a single one time administration. In some embodiments, the pharmaceutical formulation adapted for a single one time administration comprises an effective amount of ravuconazole or a derivative thereof or a prodrug thereof. In some embodiments, the effective amount of ravucanozole is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges. In some embodiments, the effective amount is such that 30 mg/kg to 50 mg/kg, preferably about 40 mg/kg is delivered to the subject in need thereof.

In certain example embodiments, the pharmaceutical formulation is adapted for oral administration. In some embodiments, the pharmaceutical formulation is adapted for topical administration. In certain example embodiments, the antifungal agent and/or pharmaceutical formulation thereof does not disrupt a microbiome in a subject in need thereof. The microbiome can be, without limitation, a gastrointestinal microbiome (including but not limited to the intestinal microbiome, stomach microbiome, and oral microbiome), skin microbiome, and/or a vaginal microbiome.

Where appropriate the compounds described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having an N. gonorrhoeae infection or a symptom thereof. In some embodiments, the subject in need thereof has or is suspected of having an N. gonorrhoeae infection or a symptom thereof, where the N. gonorrhoeae is resistant to one or more antibiotics, such as azithromycin or a derivative thereof or a prodrug thereof, penicillin or a derivative thereof or a prodrug thereof, ciprofloxacin or a derivative thereof or a prodrug thereof, ceftriaxone or a derivative thereof or a prodrug thereof, cefixime or a derivative thereof or a prodrug thereof, tetracycline or a derivative thereof or a prodrug thereof, or any combination thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount”, “effective concentration”, and/or the like refers to the amount, concentration, etc. of the primary and/or optional secondary agent included in the pharmaceutical formulation that achieve one or more therapeutic effects or desired effect. As used herein, “least effective”, “least effective concentration”, and/or the like amount refers to the lowest amount, concentration, etc. of the primary and/or optional secondary agent that achieves the one or more therapeutic or other desired effects. As used herein, “therapeutically effective amount”, “therapeutically effective concentration” and/or the like refers to the amount, concentration, etc. of the primary and/or optional secondary agent included in the pharmaceutical formulation that achieves one or more therapeutic effects. In some embodiments, the one or more therapeutic effects are to kill, inhibit the growth of, or prevent, inhibit or reduce infection by, and/or reduce the virulence or pathogenicity of Neisseria gonorrhoeae.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, pg, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments, the amount or effective amount of each of the one or more of the active agent(s) described herein contained in the pharmaceutical formulation independently ranges from about 1 pg/kg to about 100 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20^{th} ed., Lippincott Williams & Wilkins, Baltimore, MD, 2000, and “Pharmaceutical dosage forms and drug delivery systems”, 6^{th} Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof. Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives (including but not limited to antibiotics), chemotherapeutics, and combinations thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Described in certain example embodiments herein are kits comprising a pharmaceutical formulation as described herein; and instructions in a tangible medium of expression directing administration to a subject in need thereof, wherein the subject in need thereof has a Neisseria gonorrhoeae infection.

Any of the compounds, compositions, formulations, particles, cells, described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof. In some embodiments, the subject in need thereof can be in need of a treatment or prevention for a Neisseria gonorrhoeae infection.

### Methods of Treating N. Gonorrhoea

Described in certain example embodiments herein are methods of treating a Neisseria gonorrhoeae infection or a symptom thereof in a subject in need thereof, the method comprising administering, to the subject in need thereof, an amount of a triazole antifungal agent or a derivative thereof, or a prodrug thereof. In certain example embodiments, the method includes administering a pharmaceutical formulation described in greater detail elsewhere herein. In some embodiments, the method further comprises delivering a co-therapy or treatment, including but not limited to an antibiotic. Exemplary co-therapies and treatments are described in greater detail elsewhere herein, such as connection with the pharmaceutical formulations.

In certain example embodiments, the Neisseria gonorrhoeae is an antibiotic resistant Neisseria gonorrhoeae. In certain example embodiments, the Neisseria gonorrhoeae is resistant to azithromycin or a derivative thereof or a prodrug thereof, penicillin or a derivative thereof or a prodrug thereof, ciprofloxacin or a derivative thereof or a prodrug thereof, ceftriaxone or a derivative thereof or a prodrug thereof, cefixime or a derivative thereof or a prodrug thereof, tetracycline or a derivative thereof or a prodrug thereof, or any combination thereof.

In certain example embodiments, the triazole antifungal agent or a derivative thereof or a prodrug thereof, is ravuconazole or a derivative thereof or a prodrug thereof, isavuconazole or a derivative thereof or a prodrug thereof, itraconazole or a derivative thereof or a prodrug thereof, ketoconazole or a derivative thereof or a prodrug thereof, terconazole or a derivative thereof or a prodrug thereof, or any combination thereof. In some embodiments, the antifungal agent is ravuconazole or a derivative thereof or a prodrug thereof. In some embodiments, the antifungal agent is isavuconazole or a derivative thereof or a prodrug thereof. In some embodiments, the antifungal agent is itraconazole or a derivative thereof or a prodrug thereof. In some embodiments, the antifungal agent is ketoconazole or a derivative thereof or a prodrug thereof. In some embodiments, the antifungal agent is terconazole or a derivative thereof or a prodrug thereof. In some embodiments, the antifungal agents include ravuconazole or a derivative thereof or a prodrug thereof and isavuconazole or a derivative thereof or a prodrug thereof. In some embodiments, the antifungal agents include ravuconazole or a derivative thereof or a prodrug thereof and itraconazole or a derivative thereof or a prodrug thereof. In some embodiments, the antifungal agents include isavuconazole or a derivative thereof or a prodrug thereof and itraconazole or a derivative thereof or a prodrug thereof. In some embodiments, the antifungal agents include ravuconazole or a derivative thereof or a prodrug thereof, isavuconazole or a derivative thereof or a prodrug thereof, and itraconazole or a derivative thereof or a prodrug thereof.

In certain example embodiments, the amount of the amount of each of the triazole antifungal agent or a derivative thereof or a prodrug thereof independently ranges from about 10 mg to 1000 mg or more. In certain example embodiments, the amount of traizole antifungal agent or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of ravuconazole or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of isavuconazole or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of itraconazole or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of ketoconazole or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of terconazole or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of ravucanazole or a derivative thereof or a prodrug thereof ranges from about 10 mg to 500 mg. In some embodiments, the amount of ravuconazole or a derivative thereof or a prodrug thereof is any non-zero number ranging from about 0 to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 mg. In some embodiments, the amount of ravucanazole or a derivative thereof or a prodrug thereof ranges from about 50 mg to 300 mg.

In some embodiments, the amount of itraconazole or a derivative thereof or a prodrug thereof ranges from about 10 mg to 500 mg. In some embodiments, the amount of itraconazole or a derivative thereof or a prodrug thereof is any non-zero number ranging from about 0 to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 mg. In some embodiments, the amount of itraconazole or a derivative thereof or a prodrug thereof ranges from about 50 mg to 300 mg.

In some embodiments, the amount of isavuconazole or a derivative thereof or a prodrug thereof ranges from about 10 mg to 500 mg. In some embodiments, the amount of isavuconazole or a derivative thereof or a prodrug thereof is any non-zero number ranging from about 0 to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 mg. In some embodiments, the amount of isavuconazole or a derivative thereof or a prodrug thereof ranges from about 50 mg to 300 mg.

In some embodiments, the amount of ketoconazole or a derivative thereof or a prodrug thereof ranges from about 10 mg to 500 mg. In some embodiments, the amount of ketoconazole or a derivative thereof or a prodrug thereof is any non-zero number ranging from about 0 to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 mg. In some embodiments, the amount of ketoconazole or a derivative thereof or a prodrug thereof ranges from about 50 mg to 300 mg.

In some embodiments, the amount of terconazole or a derivative thereof or a prodrug thereof ranges from about 10 mg to 500 mg. In some embodiments, the amount of terconazole or a derivative thereof or a prodrug thereof is any non-zero number ranging from about 0 to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 mg. In some embodiments, the amount of terconazole or a derivative thereof or a prodrug thereof ranges from about 50 mg to 300 mg.

In certain example embodiments, the pharmaceutical formulation comprises an amount of an antibiotic or a derivative thereof or a prodrug thereof. In some embodiments, the antibiotic is one effective to treat N. gonorrhoeae infection. In certain example embodiments, the antibiotic is azithromycin or a derivative thereof or a prodrug thereof, penicillin or a derivative thereof or a prodrug thereof, ciprofloxacin or a derivative thereof or a prodrug thereof, ceftriaxone or a derivative thereof or a prodrug thereof, cefixime or a derivative thereof or a prodrug thereof, tetracycline or a derivative thereof or a prodrug thereof, or any combination thereof.

In certain example embodiments, the amount of the antibiotic is about 0.001 M to Ig or more. In some embodiments, the amount of antibiotic or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of azithromycin or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of penicillin or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of ciprofloxacin or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of ceftriaxone or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of cefixime or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the amount of tetracycline or a derivative thereof or a prodrug thereof is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, only one dose of the pharmaceutical formulation is effective to treat the N. gonorrhoeae infection. As such in certain example embodiments, the pharmaceutical formulation is adapted for a single one time administration. In some embodiments, the pharmaceutical formulation adapted for a single one time administration comprises an effective amount of ravuconazole or a derivative thereof or a prodrug thereof. In some embodiments, the effective amount of ravucanozole is any non-zero number ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges. In some embodiments, the effective amount is such that 30 mg/kg to 50 mg/kg, preferably about 40 mg/kg is delivered to the subject in need thereof.

The method can include administration of the pharmaceutical formulations or dosage forms thereof described herein one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly) to the subject in need thereof. In some embodiments, the method includes continuous administration over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the method can include administering a predetermined amount that is an appropriate fraction of the effective amount of the active ingredient. Such unit doses may therefore be administered once or more than once a day, month, or year (e.g., 1, 2, 3, 4, 5, 6, or more times per day, month, or year).

Where co-therapies or multiple pharmaceutical formulations are to be delivered to a subject, the method can include administration of different therapies or formulations can sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

In some embodiments administration of the triazole antifungal, or a derivative thereof, or a prodrug thereof is oral. Other appropriate routes of administration are described elsewhere herein. In some embodiments, administration of a co-therapeutic antibiotic is oral or via injection. Other appropriate routes of administration of a co-therapeutic antibiotic are described elsewhere herein.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Treatment of Neisseria gonorrhoeae with Ravuconazole

N. gonorrhoeae represents an urgent public health threat due to the rapid emergence of resistance to current antibiotics and the limited number of anti-gonococcal agents currently in clinical trials (1). Neisseria gonorrhoeae is the second most reported notifiable disease in the United States and one of the most important antimicrobial resistance threats worldwide (2). The CDC has listed drug-resistant N. gonorrhoeae at the highest possible threat level to public health. With no effective vaccines available, the development of effective antibacterial agents is a critical priority.

Few effective therapeutic options are available to treat gonorrhea. Current guidelines recommend treating gonorrhea with injectable ceftriaxone. However, increasing resistance to this treatment option has been reported, which has resulted in multidrug-resistant N. gonorrhoeae (i.e., super gonorrhea) becoming a critical public health concern. Compounding the problem further is that over the last 3 decades, almost no new classes of antibiotic drugs have been approved, raising a serious concern that super gonorrhea infections may be untreatable in the near future. (1-7).

Drug development is a time-consuming, costly, and high-risk venture given few compounds successfully make it through stringent regulatory requirements to the marketplace. Collectively, this points to a critical need for the identification of novel strategies to develop antibiotics to deal with this challenging health issue. One strategy which warrants more attention for identifying new antimicrobials is drug repurposing (8).

Recently, this strategy is gaining momentum as it has resulted in successes in a number of disease areas and has accounted for approximately 30% of newly approved FDA drugs and vaccines (9-11). Repurposing existing approved drugs permits companies to bypass much of the preclinical work and early stage clinical trials required for new compounds (particularly toxicological and pharmacological analysis of drugs) thus cutting into the cost associated with bringing a drug to the marketplace (12).

In an intensive search for antimicrobial activity among non-antibiotic FDA approved drugs and clinical safe molecules, Applicant recently studied several drugs for N. gonorrhoeae (1-4, 6, 7). Applicant has identified ravuconazole and isavuconazole as the most promising new candidates for the treatment of drug-resistant gonorrhea infections. Ravuconazole (BMS-207147; ER-30346) is an antifungal triazole that was discovered in the late 1990s (13). There was a clinical trial in 2003 (NCT00064311) but the drug was not approved in USA probably due to availability of other potent antifungal drugs. Ravuconazole is approved in Japan for treatment of onychomycosis (NAILIN) in 2018. 1 capsule (100 mg as ravuconazole) administered orally once daily for a period of at least 12 weeks. There are currently several ongoing clinical trials for neglected diseases in Africa.

This Example demonstrates at least that a non-antibacterial, such as ravuconazole, is effective against Neisseria gonorrhoeae (N. gonorrhoeae). Ravuconazole has at least these advantages as compared to the gold standard treatments for N. gonorrhoeae. First it is orally bioavailable (current drug of choice, ceftriaxone, is injectable). Second, ravuconazole doesn't interfere with normal microbiome (e.g., vaginal and intestinal microbiome). Ceftriaxone disturbs both. Third, potent activity against N. gonorrhoeae is achievable in a clinical dose range. Fourth, ravuconazole is active against isolates resistant to drugs of choice azithromycin and Ceftriaxone. Fifth, no pre-existing resistance was found to ravuconazole. Sixth ravuconazole has an exceptional pharmacokinetic profile and tissue distribution (up to 8 days half-life from single dose). Seventh ravuconazole is effective with single dose clearance (required for patient compliance for Neisseria treatment). Finally, safety and toxicity in humans already established for ravuconazole. Overall, Applicants at least demonstrate that ravuconazole is superior to current drug therapies for N. gonnorrhoeae such as azithromycin and ceftriaxone and to any drug currently in clinical trial (e.g., Zoliflodacin, and Gepotidacin) for N. gonnorrhoeae.

### Identification of Ravuconazole.

To identify drugs capable of killing Neisseria gonorrhoeae, the Johns Hopkins Clinical Compounds Library (1514), the MCE FDA-Approved Drug Library (2441), and the Pharmakon library (1600) were screened against N. gonorrhoeae. Positive hits that are capable of killing Neisseria gonorrhoeae in clinical range were identified (itraconazole, ravuconazole and isavuconazole).

The most potent hit identified was (Ravuconazole), antifungal identified in late 1990s and currently approved in Japan for treatment of Onychomycosis (toe nail fungus).

### Susceptibility Analysis of Ravuconazole Against Clinical Isolates ofN. Gonorrhoeae.

The anti-gonococcal activity of Ravuconazole against a panel of 84 N. gonorrhoeae clinical isolates was identified. Ravuconazole inhibited growth of all N. gonorrhoeae strains tested at concentrations ranging from 0.008 μg/mL up to 0.125 μg/mL with MIC50 and MIC90 values of 0.03 μg/mL and 0.06 μg/mL, respectively (Table 1). This activity was more potent than current drugs of choice for treatment of N. gonnorrhoeae (e.g., Ceftriaxone or Azithromycin).

TABLE 1
Summary of MIC_{50/90} against 104 N. gonorrhoeae isolates

| Drugs | MIC₅₀ | MIC₉₀ | Range |
|---|---|---|---|
| Itraconazole | 1 | 2 | 0.03-4 |
| Isavuconazole | 0.5 | 1 | 0.015-2 |
| Ravuconazole | 0.03 | 0.06 | 0.002-0. 25 |
| Azithromycin | 1 | 4 | 0.03-512 |
| Ceftriaxone | 0.03 | 0.06 | 0.001-2 |
| Zoliflodacin | 0.06 | 0.125 | 0.004-0.5 |
| Gepotidacin | 0.25 | 0.5 | 0.03-8 |

Ravuconazole Doesn't Interfere with Vaginal Microbiome or Intestinal Microbiome

N. gonorrhoeae pathogenesis greatly depends on colonization of the reproductive tract. One of the natural barriers of such colonization is an intact healthy microbiome. Species of lactobacilli present in the genitourinary tract play a significant role in hindering colonization by Neisseria(1). Thus, Applicant explored the antibacterial activity of ravuconazole against commensal vaginal lactobacilli strains. As presented in Table 2, ravuconazole inactive against all strains of lactobacilli (MIC>256 μg/mL), suggesting that they could be used against gonococci without disrupting the normal microbiota. Conversely, azithromycin and ceftriaxone the drug of choice, inhibited the tested normal microbiota strains.

TABLE 2
The minimum inhibitory concentration (MIC in μg/mL) against commensal
Lactobacillus strains from the female reproductive tract.

| | Bacterial | | | | | |
|---|---|---|---|---|---|---|
| No. | strains | Itraconazole | Isavuconazole | Ravuconazole | Azithromycin | Ceftriaxone |
| 1 | L. | >256 | >256 | >256 | 1 | 2 |
| | crispatus | | | | | |
| | HM-103 | | | | | |
| 2 | L. | >256 | >256 | >256 | 2 | 1 |
| | crispatus | | | | | |
| | HM-371 | | | | | |
| 3 | L. | >256 | >256 | >256 | 1 | 4 |
| | crispatus | | | | | |
| | HM-638 | | | | | |
| 4 | L. gasseri | >256 | >256 | >256 | ≤0.5 | ≤0.5 |
| | HM-407 | | | | | |
| 5 | L. gasseri | >256 | >256 | >256 | 2 | 2 |
| | HM-642 | | | | | |
| 6 | L. gasseri | >256 | >256 | >256 | 2 | 1 |
| | HM-644 | | | | | |
| 7 | L. gasseri | >256 | >256 | >256 | 1 | 1 |
| | HM-647 | | | | | |
| 8 | L. jensenii | >256 | >256 | >256 | ≤0.5 | ≤0.5 |
| | HM-105 | | | | | |
| 9 | L. jensenii | >256 | >256 | >256 | 1 | ≤0.5 |
| | HM-372 | | | | | |
| 10 | L. jensenii | >256 | >256 | >256 | 1 | 1 |
| | HM-374 | | | | | |
| 11 | L. jensenii | >256 | >256 | >256 | 1 | ≤0.5 |
| | HM-639 | | | | | |
| 12 | L. jensenii | >256 | >256 | >256 | ≤0.5 | ≤0.5 |
| | HM-640 | | | | | |
| 13 | L. jensenii | >256 | >256 | >256 | 1 | ≤0.5 |
| | HM-645 | | | | | |
| 14 | L. jensenii | >256 | >256 | >256 | 2 | ≤0.5 |
| | HM-646 | | | | | |
| 15 | L. | >256 | >256 | >256 | 2 | 2 |
| | johnsonii | | | | | |
| | HM-643 | | | | | |

Efficacy of Ravuconazole Single Oral Dose in a Mouse Model of Neisseria gonorrhoeae Genital Tract Infection.

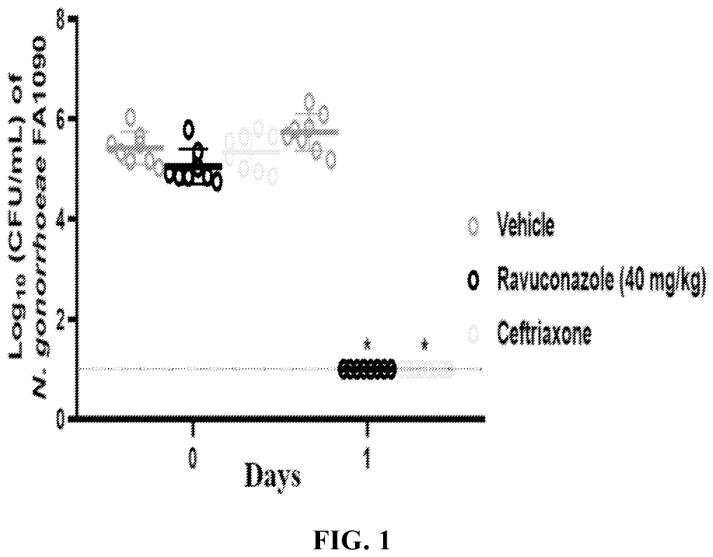

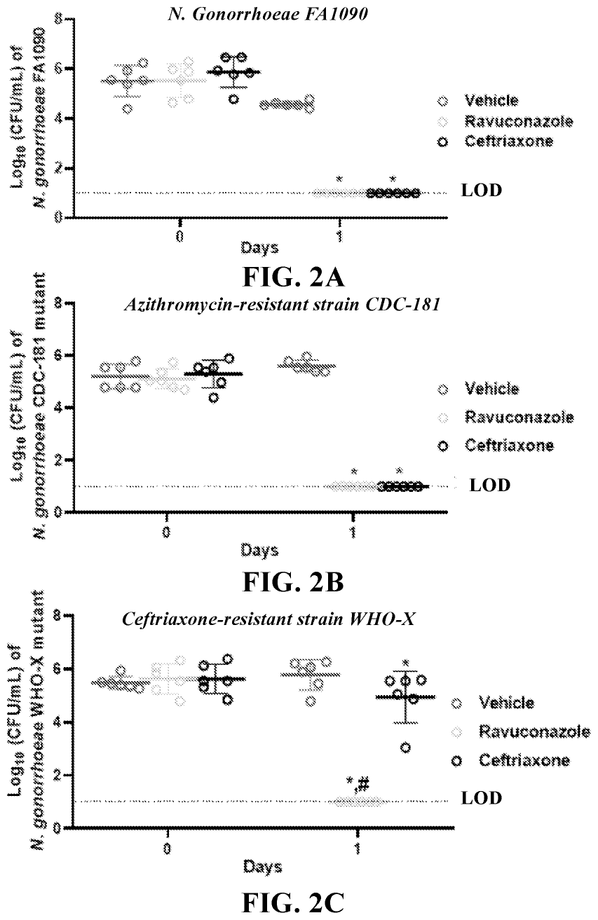

The in vivo activity of ravuconazole against N. gonorrhoeae FA1090, azithromycin resistant N. gonorrhoeae CDC-181, and ceftriaxone-resistant N. gonorrhoeae WHOAX was evaluated using a female mouse model of N. gonorrhoeae genital tract infection, as previously described (3, 4). Mice were treated with Ravuconazole single dose (40 mg/kg) orally for N. gonorrhoeae FA1090, and single dose (160 mg/kg) orally for azithromycin resistant N. gonorrhoeae CDC-181 and ceftriaxone-resistant N. gonorrhoeae WHO-X (fosravuconazole Prodrug). Fosravuconazole Prodrug was delivered via the oral route because this is the normal route of administration for humans. As shown in FIG. 1, and FIG. 2A-2C treatment of mice with Ravuconazole cleared infection. Remarkably, a single oral dose of ravuconazole cleared the multidrug-resistant strain WHO-X, while the injectable ceftriaxone failed to clear the infection as reported before (14).

Efficacy of Ravuconazole Single Oral Doses in a Mouse Model of Neisseria gonorrhoeae Genital Tract Infection.

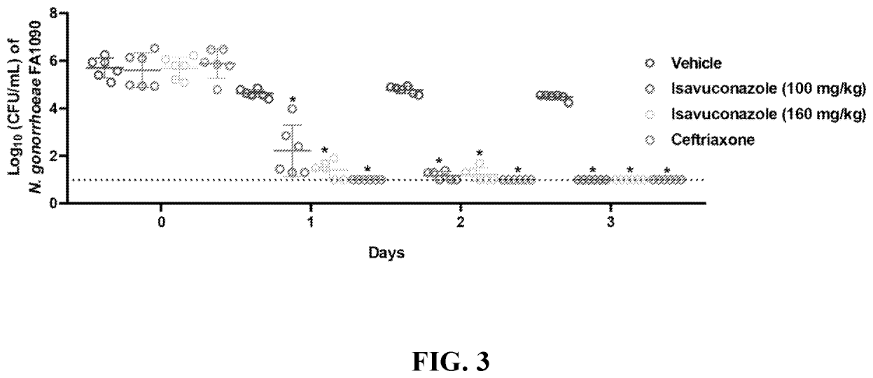

Encouraged by results of ravuconazole Applicant tested efficacy of isavuconazole (100 mg/kg and 160 mg/kg) orally for 3 consecutive days in a mouse model against N. gonorrhoeae FA1090. As shown in FIG. 3, treatment of mice with isavuconazole cleared infection by the third day. Remarkably, the low dose (100 mg/kg) of isavuconazole cleared the infection in 50% of the infected mice on the second day and in 100% of the mice by the third day of treatment (FIG. 3).

### REFERENCES FOR EXAMPLE 1

1. Elkashif A, Seleem M N. Investigation of auranofin and gold-containing analogues antibacterial activity against multidrug-resistant Neisseria gonorrhoeae. Sci Rep. 2020; 10(1):5602.
2. Alhashimi M, Mayhoub A, Seleem M N. Repurposing salicylamide for combating multidrug-resistant Neisseria gonorrhoeae. Antimicrob Agents Chemother. 2019.
3. Elhassanny A E M, Abutaleb N S, Seleem M N. Auranofin exerts antibacterial activity against Neisseria gonorrhoeae in a female mouse model of genital tract infection. PLoS One. 2022; 17(4):e0266764.
4. Abutaleb N S, Elhassanny A E M, Seleem M N. In vivo efficacy of acetazolamide in a mouse model of Neisseria gonorrhoeae infection. Microb Pathog. 2022; 164:105454.
5. Naclerio G A, Abutaleb N S, Alhashimi M, Seleem M N, Sintim H O. N-(1,3,4-Oxadiazol-2-yl)Benzamides as Antibacterial Agents against Neisseria gonorrhoeae. Int J Mol Sci. 2021; 22(5).
6. Hewitt C S, Abutaleb N S, Elhassanny A E M, Nocentini A, Cao X, Amos D P, et al. Structure-Activity Relationship Studies of Acetazolamide-Based Carbonic Anhydrase Inhibitors with Activity against Neisseria gonorrhoeae. ACS Infect Dis. 2021; 7(7):1969-84.
7. Seong Y J, Alhashimi M, Mayhoub A, Mohammad H, Seleem M N. Repurposing Fenamic Acid Drugs To Combat Multidrug-Resistant Neisseria gonorrhoeae. Antimicrob Agents Chemother. 2020; 64(7).
8. Thangamani S, Mohammad H, Younis W, Seleem M N. Drug repurposing for the treatment of staphylococcal infections. Current pharmaceutical design. 2015; 21(16):2089-100.
9. Jin G, Wong S T. Toward better drug repositioning: prioritizing and integrating existing methods into efficient pipelines. Drug discovery today. 2013.
10. Ashburn T T, Thor K B. Drug repositioning: identifying and developing new uses for existing drugs. Nature reviews Drug discovery. 2004; 3(8):673-83.
11. Chong C R, Sullivan D J, Jr. New uses for old drugs. Nature. 2007; 448(7154):645-6.
12. DiMasi J A, Hansen R W, Grabowski H G. The price of innovation: new estimates of drug development costs. Journal of health economics. 2003; 22(2):151-85.
13. Fung-Tomc J C, Huczko E, Minassian B, Bonner D P. In vitro activity of a new oral triazole, BMS-207147 (ER-30346). Antimicrob Agents Chemother. 1998; 42(2):313-8.
14. Connolly, K. L., et al., Pharmacokinetic Data Are Predictive of In Vivo Efficacy for Cefixime and Cefriaxone against Susceptible and Resistant Neisseria gonorrhoeae Strains in the Gonorrhea Mouse Model. Antimicrob Agents Chemother, 2019. 63(3)

### Example 2

### Identification of a Novel Class of Anti-Gonococcal Agents

To address the need for novel strategies to combat the growing problem of N. gonorrhoeae, we screened FDA-approved drugs and clinical molecules in search of novel drugs that possess potent and specific anti-gonococcal activity against multidrug-resistant N. gonorrhoeae. Several promising hits were identified and evaluated successfully both in vitro and in vivo by our group [1-9]. The most potent and novel hits that displayed exceptional activity were the azole drugs itraconazole, isavuconazole, and ravuconazole. In vitro activity was determined against 103 N. gonorrhoeae isolates with different resistance profiles (Table 3). Ravuconazole exhibits MIC50 and MIC90 values of 0.03 μg/mL and 0.06 μg/mL, respectively, which is similar to ceftriaxone. The MIC range for ravuconazole (0.002-0.25 μg/mL) was better than ceftriaxone (0.001-2 μg/mL). The highest MIC value for ravuconazole was 0.25 μg/mL, which was less than the value for ceftriaxone (2 μg/mL). Isavuconazole exhibits MIC50 and MIC90 values of 0.5 μg/mL and 1 μg/mL, respectively, which is similar to gepotidacin. The MIC range for isavuconazole (0.015-4 μg/mL) was better than gepotidacin (0.03-8 μg/mL). Itraconazole was less potent with MIC50 and MIC90 values of 1 μg/mL and 2 μg/mL, respectively, which was less than the value for azithromycin. The MIC range for itraconazole (0.03-8 μg/mL) was similar to gepotidacin (0.03-8 μg/mL). MICs of ravuconazole and control antibiotics against 7 resistant N. gonorrhoeae isolates are shown in Table 4.

TABLE 3
Summary of MIC_{50/90} values (μg/mL)
against 103 N. gonorrhoeae isolates

| Drugs | MIC₅₀ | MIC₉₀ | Range |
|---|---|---|---|
| Itraconazole | 1 | 2 | 0.03-8 |
| Isavuconazole | 0.5 | 1 | 0.015-4 |
| Ravuconazole | 0.03 | 0.06 | 0.002-0.25 |
| Ceftriaxone | 0.03 | 0.06 | 0.001-2 |
| Azithromycin | 1 | 4 | 0.03-512 |
| Zoliflodacin | 0.06 | 0.125 | 0.004-0.25 |
| Gepotidacin | 0.5 | 1 | 0.03-8 |

TABLE 4
MICs of ravuconazole and control antibiotics against
resistant N. gonorrhoeae isolates by agar dilution

| N. gonorrhoeae | | | |
|---|---|---|---|
| strains | Ravuconazole | Ceftriaxone | Azithromycin |
| WHO-L | 0.25 | 0.5 | 0.5 |
| WHO-X | 0.25 | 2 | 0.5 |
| WHO-Y | 0.25 | 1 | 1 |
| WHO-Z | 0.25 | 0.5 | 1 |
| CDC-179 | 0.06 | 0.015 | 8 |
| CDC-181 | 0.25 | 0.06 | 256 |
| CDC-174 | 0.25 | 0.25 | 1 |

Azoles Exhibit Bactericidal Activity Against N. gonorrhoeae:

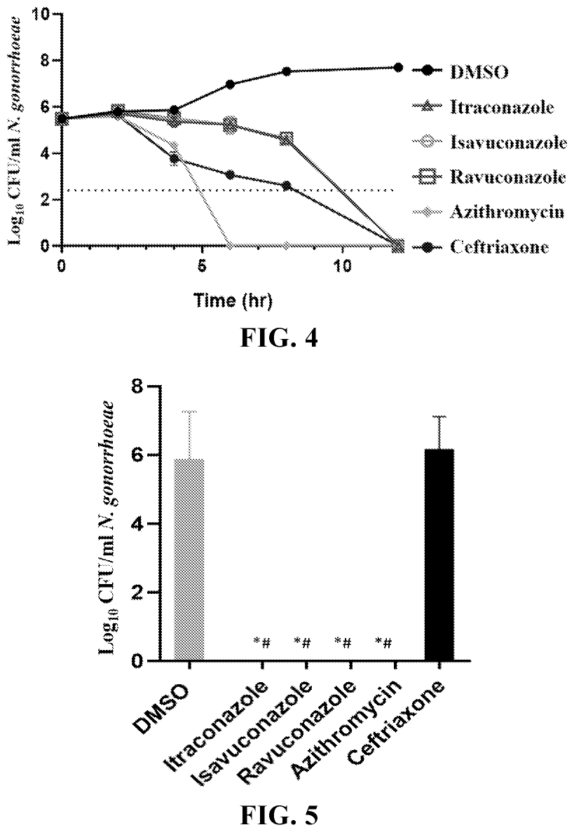

To examine the killing kinetics of azoles against N. gonorrhoeae, we examined whether these agents exhibit bacteriostatic or bactericidal activity via a standard time-kill assay [3-5] against 3 strains of N. gonorrhoeae. The starting inoculum of ˜5×10⁵ CFU/mL N. gonorrhoeae FA1090 was reduced to the limit of detection within 12 h of treatment (data for 1 strain N. gonorrhoeae FA1090 is shown in FIG. 4) indicating a bactericidal activity.

### Frequency of Spontaneous Mutation:

Using the single-step resistance assay [3-5], we examined the likelihood that N. gonorrhoeae will develop resistance to itraconazole, isavuconazole, and ravuconazole. No resistant mutants were isolated at 10×MIC, which corresponds to a resistance frequency <1.64×10⁻¹⁰.

Azoles Clear Intracellular N. gonorrhoeae

N. gonorrhoeae can invade and replicate inside mucosal epithelial cell layers in the urogenital tract, rectum, and pharynx. N. gonorrhoeae is also capable of transmigrating across the mucosal epithelia following invasion and can cause disseminated infections [10-12]. Although ceftriaxone is used clinically to treat gonococcal infections, it is unable to clear intracellular N. gonorrhoeae due to its high polarity and poor cellular permeability [1, 3, 5]. Alternative drugs with the ability to penetrate infected cells to clear the infection are a major advantage. We used the gentamicin protection assay [3-5, 10, 11] to examine the ability of azoles to reduce the burden of intracellular N. gonorrhoeae present in infected endocervical cells. Endocervical cells (End1/E6E7) were infected with N. gonorrhoeae FA1090 and subsequently treated for 24 h at 3×MIC with either itraconazole, isavuconazole, ravuconazole, azithromycin, or ceftriaxone. As presented in FIG. 5, the azoles were superior to ceftriaxone and cleared intracellular N. gonorrhoeae FA1090 within infected endocervical cells.

Post-Antibiotic Effect (PAE) of Ravuconazole Against N. gonorrhoeae.

Determining the PAE of a new antimicrobial is an important step for dosing and has clinical importance [13-15]. Antimicrobials with an extended PAE possess several advantages, including reduced costs of the drug, limited toxicity, and better patient compliance [15-17]. To investigate the ability of azoles to exhibit a prolonged inhibitory effect against N. gonorrhoeae following a brief exposure period (1 h at 10×MIC), a PAE experiment was performed [3-5]. Ravuconazole and isavuconazole exhibited a long PAE that ranged from 10 to >16 h, which was longer than both ceftriaxone and azithromycin (Table 5). The PAE for gepotidacin against N. gonorrhoeae was reported to range from 0.5 to >2.5 h [18] while the PAE for zoliflodacin against Staphylococcus aureus reported to be 1.65 to 2.4 h [19]. These results provide valuable evidence that Neisseria is very slow to recover after exposure to ravuconazole with irreversible potent target engagement.

TABLE 5
Post-antibiotic effect of itraconazole (ITC), isavuconazole (ISA),
ravuconazole (RAV), ceftriaxone (CEF), and azithromycin (AZM)

| Ng | PAE (hours) | | | | |
|---|---|---|---|---|---|
| Strains | ITC | ISA | RAV | CEF | AZM |
| FA 1090 | 4 | 10 | 12 | 4 | 6 |
| WHO Y | 4 | 10 | 16 | 6 | 4 |
| WHO P | 4 | 10 | >16 | 8 | 8 |

### Azoles do not Inhibit Growth of Healthy Vaginal Microflora

A healthy vaginal microbiome that is dominated by Lactobacillus spp. is less susceptible to N. gonorrhoeae infection and other sexually transmitted diseases (STDs) [20-25]. However, a disadvantage with all drugs currently used to treat N. gonorrhoeae infections, including drugs in clinical trials, is that they are nonspecific and inhibit growth ofboth gonococci andbeneficial commensal bacteria indiscriminately (Table 6). Identifying novel drugsthat protect healthy microflora while selectively killing Neisseria spp. would ideally result in a better post-treatment outcome. We tested the activity of azoles against important members of healthy vaginal microbiota that inhibit N. gonorrhoeae colonization. The azoles were not active against all species of Lactobacillus tested, with MIC values >256 μg/mL (Table 6). In contrast, ceftriaxone, azithromycin, zoliflodacin, and gepotidacin inhibited the growth of all strains of Lactobacillus tested (MVIC was <1 μg/mL).

TABLE 6
MIC of itraconazole (ITC), isavuconazole (ISA), ravuconazole
(RAV), ceftriaxone (CEF), azithromycin (AZM), zoliflodacin (ZOL),
and gepotidacin (GEP) against vaginal microflora strains.

| Species of | | | | | | | |
|---|---|---|---|---|---|---|---|
| Lactobacillus | MIC Range (μg/mL) | | | | | | |
| (N. of strains) | ITC | ISA | RAV | CEF | AZM | ZOL | GEP |
| L. crispatus | ≥256 | ≥256 | ≥256 | ≤1-2 | ≤1 | ≤1 | ≤1 |
| (3) | | | | | | | |
| L. gasseri (4) | ≥256 | ≥256 | ≥256 | ≤1 | ≤1 | ≤1 | ≤1 |
| L. jensenii | ≥256 | ≥256 | ≥256 | ≤1-2 | ≤1 | ≤1 | ≤1 |
| (7) | | | | | | | |
| L. johsonii | ≥256 | ≥256 | ≥256 | ≤1 | ≤1 | ≤1 | ≤1 |
| (2) | | | | | | | |

### Azoles do not Disrupt the Intestinal Microbiota

Disrupting the intestinal microbiota using broad-spectrum antimicrobial therapy increases the susceptibility of hosts to C. difficile colonization [26-33]. One of the reasons the CDC changed the recommendation from dual therapy (ceftriaxone plus azithromycin) treatment to ceftriaxone alone for N. gonorrhoeae was a concern regarding potential harm of dual therapy on the microbiome [34]. Thus, it is important for new anti-gonococcal drugs to show minimal activity against the normal microbiome especially if the drug will be combined clinically with other broad-spectrum drugs, such as azithromycin or ceftriaxone. Unlike ceftriaxone, azithromycin, zoliflodacin, and gepotidacin, the azoles exhibited no activity against representative members of the human normal intestinal microbiota (Table 7). In contrast, a clinical trial found there was a significant change in the microbiome diversity of the gastrointestinal tract (GIT) and the vagina in patients who took gepotidacin orally for 5 days [35]. These results confirm the specificity and uniqueness of azoles and their potential application for dual therapy.

TABLE 7
MIC of (ITC), (ISA), (RAV), (CEF), (AZM), (ZOL),
and (GEP) against normal gut microbiota.

| Species | MIC Range (μg/mL) | | | | | | |
|---|---|---|---|---|---|---|---|
| (No. of strains) | ITC | ISA | RAV | CEF | AZM | ZOL | GEP |
| Bacteroides | >256 | >256 | >256 | 2-≥16 | 0.6-≥16 | 2-8 | 0.25-2 |
| fragilis (4) | | | | | | | |
| Bacteroides | >256 | >256 | >256 | 4-16 | 4-16 | 0.5-1 | 0.125 |
| dorei (2) | | | | | | | |
| Bacteroides | >256 | >256 | >256 | 1-4 | 0.03-0.06 | 4 | <0.125 |
| breve (2) | | | | | | | |
| Bacteroides | >256 | >256 | >256 | 0.125 | 0.015 | 1 | <0.125 |
| adolescentis (2) | | | | | | | |
| Bacteroides | >256 | >256 | >256 | 4 | 0.25 | 1-2 | 0.5 |
| longum (2) | | | | | | | |
| Lactobacillus | >256 | >256 | >256 | 16 | 0.5 | <0.125 | 0.25 |
| casei (1) | | | | | | | |
| Lactobacillus | >256 | >256 | >256 | 0.5 | 1 | 1 | 0.5 |
| brevis (1) | | | | | | | |
| Bifidobacterium | >256 | >256 | >256 | 0.125 | 0.03 | 1 | 0.125 |
| angulatum (1) | | | | | | | |
| Lactobacillus | >256 | >256 | >256 | 16 | 1 | 0.125 | 0.5 |
| rhamnosus (1) | | | | | | | |
| Escherichia coli | >256 | >256 | >256 | 0.03 | 2 | 0.25 | 0.125 |
| (1) | | | | | | | |
| Enterococcus | >256 | >256 | >256 | 8 | 2 | 0.5 | 1 |
| faecalis (1) | | | | | | | |
| Enterococcus | >256 | >256 | >256 | 8 | 0.25 | 2 | 2 |
| faecium (1) | | | | | | | |
| Staphylococcus | >256 | >256 | >256 | 2 | 0.25 | 0.125 | 0.25 |
| aureus (1) | | | | | | | |

### The Importance of Fluoro Substitution on the Phenyl Ring

Ravuconazole (1) is similar in structure to isavuconazole (2) with only a fluoro substitution on the phenyl ring. However, ravuconazole exhibits a ≥125-fold more potent antimicrobial activity than isavuconazole (Table 3 and Table 8). Without being bound by theory Applicant hypothesized that the position of 1 fluorine could account for ravuconazole's superior antimicrobial activity.

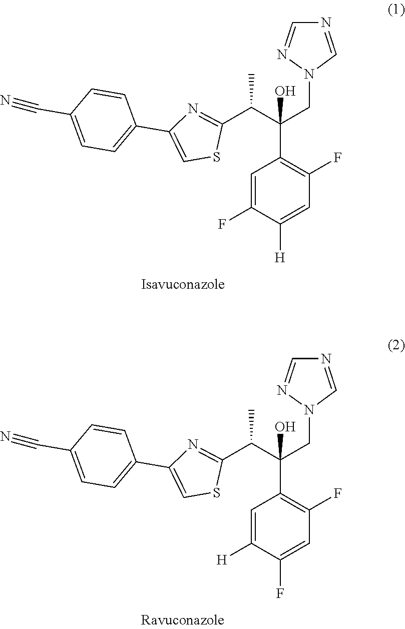 [SMILES: C[C@@H](c1nc(-c2ccc(C#N)cc2)cs1)[C@](O)(Cn1cncn1)c1cc(F)ccc1F ; C[C@@H](c1nc(-c2ccc(C#N)cc2)cs1)[C@](O)(Cn1cncn1)c1ccc(F)cc1F]

TABLE 8
MIC of itraconazole (ITC), isavuconazole (ISA),
ravuconazole (RAV), ceftriaxone (CEF), and azithromycin
(AZM) against Neisseria spp.

| Species (No. | MIC Range (μg/mL) | | | | |
|---|---|---|---|---|---|
| of Strains) | ITC | ISA | RAV | CEF | AZM |
| N. | 0.125-1 | 0.25-0.5 | 0.004-0.008 | 0.004-0.008 | 0.125-1 |
| meingitidis (5) | | | | | |
| N. | 64 | 16 | 0.125 | 0.125 | 2 |
| flavescens (1) | | | | | |
| N. mucosa (1) | 64 | 8 | 0.125 | 0.125 | 2 |
| N. | 4 | 1 | 0.03 | 0.125 | 2 |
| lactamica (1) | | | | | |

### Target Specificity for Azoles.

Intrigued by the specificity and potent activity of azoles against N. gonorrhoeae, we evaluated the extent of the azoles' antibacterial activity against other Neisseria species (Table 8). The azoles exhibit potent activity against N. meningitidis. Ravuconazole exhibited the most potent activity against all Neisseria spp. including N. flavescens, N. mucosa, and N. lactamica. The potent activity of ravuconazole and isavuconazole against N. meningitidis is of special interest and will allow a less frequent (once a week) therapeutic regimen (orally or IV), once a week, which is needed in sub-Saharan Africa for patients with limited access to proper healthcare facilities [36-40].

### REFERENCES FOR EXAMPLE 2

1. Elhassanny, A. E. M., N. S. Abutaleb, and M. N. Seleem, Auranofin exerts antibacterial activity against Neisseria gonorrhoeae in a female mouse model of genital tract infection. PLoS One, 2022. 17(4): p. e0266764.
2. Abutaleb, N. S., A. E. M. Elhassanny, and M. N. Seleem, In vivo efficacy of acetazolamide in a mouse model of Neisseria gonorrhoeae infection. Microb Pathog, 2022. 164: p. 105454.
3. Seong, Y. J., et al., Repurposing Fenamic Acid Drugs To Combat Multidrug-Resistant Neisseria gonorrhoeae. Antimicrob Agents Chemother, 2020. 64(7).
4. Elkashif, A. and M. N. Seleem, Investigation of auranofin and gold-containing analogues antibacterial activity against multidrug-resistant Neisseria gonorrhoeae. Sci Rep, 2020. 10(1): p. 5602.
5. Alhashimi, M., A. Mayhoub, and M. N. Seleem, Repurposing salicylamide for combating multidrug-resistant Neisseria gonorrhoeae. Antimicrob Agents Chemother, 2019.
6. Marapaka, A. K., et al., Structural Characterization of Thiadiazolesulfonamide Inhibitors Bound to Neisseria gonorrhoeae alpha-Carbonic Anhydrase. ACS Med Chem Lett, 2023. 14(1): p. 103-109.
7. Giovannuzzi, S., et al., Dithiocarbamates effectively inhibit the alpha-carbonic anhydrasefrom Neisseria gonorrhoeae. J Enzyme Inhib Med Chem, 2022. 37(1): p. 1-8.
8. Abutaleb, N. S., et al., Repurposing FDA-approved sulphonamide carbonic anhydrase inhibitors for treatment of Neisseria gonorrhoeae. J Enzyme Inhib Med Chem, 2022. 37(1): p. 51-61.
9. Hewitt, C. S., et al., Structure-Activity Relationship Studies of Acetazolamide-Based Carbonic Anhydrase Inhibitors with Activity against Neisseria gonorrhoeae. ACS Infect Dis, 2021. 7(7): p. 1969-1984.
10. Lu, P., et al., A Subpopulation of Intracellular Neisseria gonorrhoeae Escapes Autophagy-Mediated Killing Inside Epithelial Cells. J Infect Dis, 2019. 219(1): p. 133-144.
11. Solger, F., et al., A Role of Sphingosine in the Intracellular Survival of Neisseria gonorrhoeae. Front Cell Infect Microbiol, 2020. 10: p. 215.
12. Post, D. M., et al., Intracellular survival of Neisseria gonorrhoeae in male urethral epithelial cells: importance of a hexaacyl lipid A, Infect Immun, 2002. 70(2): p. 909-20.
13. Vogelman, B., et al., In vivo postantibiotic effect in a thigh infection in neutropenic mice. J Infect Dis, 1988. 157(2): p. 287-98.
14. Craig, W. A. and B. Vogelman, The post antibiotic effect. Ann Intern Med, 1987. 106(6): p. 900-2.
15. Craig, W. A., Pharmacokinetic/pharmacodynamic parameters: rationale for antibacterial dosing of mice and men. Clin Infect Dis, 1998. 26(1): p. 1-10; quiz 11-2.
16. Mohammad, H., et al., Antibacterial Characterization of Novel Synthetic Thiazole Compounds against Methicillin-Resistant Staphylococcus pseudintermedius. PLoS One, 2015. 10(6): p. e0130385.
17. Levison, M. E., Pharmacodynamics of antimicrobial drugs. Infect Dis Clin North Am, 2004. 18(3): p. 451-65, vii.
18. Lass-Florl, C., Triazole antifungal agents in invasive fungal infections: a comparative review. Drugs, 2011. 71(18): p. 2405-19.
19. Bradford, P. A., et al., Zoliflodacin: An Oral Spiropyrimidinetrione Antibiotic for the Treatment of Neisseria gonorrheae, Including Multi-Drug-Resistant Isolates. ACS Infect Di s, 2020. 6(6): p. 1332-1345.
20. Dimitonova, S. P., et al., Antimicrobial activity and protective properties of vaginal lactobacilli from healthy Bulgarian women. Anaerobe, 2007. 13(5-6): p. 178-84.
21. Breshears, L. M., et al., Lactobacillus crispatus inhibits growth of Gardnerella vaginalis and Neisseria gonorrhoeae on a porcine vaginal mucosa model. BMC Microbiol, 2015. 15: p. 276.
22. Antonio, M. A., S. E. Hawes, and S. L. Hillier, The identification of vaginal Lactobacillus species and the demographic and microbiologic characteristics of women colonized by these species. J Infect Dis, 1999. 180(6): p. 1950-6.
23. Hillier, S. L., et al., The relationship of hydrogen peroxide-producing lactobacilli to bacterial vaginosis and genital microflora in pregnant women. Obstet Gynecol, 1992. 79(3): p. 369-73.
24. Ravel, J., et al., Vaginal microbiome of reproductive-age women. Proc Natl Acad Sci USA, 2011. 108 Suppl 1: p. 4680-7.
25. Barrons, R. and D. Tassone, Use of Lactobacillus probiotics for bacterial genitourinary infections in women: a review. Clin Ther, 2008. 30(3): p. 453-68.
26. Abutaleb, N. S. and M. N. Seleem, Repurposing the Antiamoebic Drug Diiodohydroxyquinoline for Treatment of Clostridioides difficile Infections. Antimicrob Agents Chemother, 2020. 64(6).
27. AbdelKhalek, A. and M. N. Seleem, Repurposing the Veterinary Antiprotozoal Drug Ronidazole for the Treatment of Clostridioides difficile Infection. Int J Antimicrob Agents, 2020. 56(6): p. 106188.
28. Pal, R. and M. N. Seleem, Discovery of a novel natural product inhibitor of Clostridioides difficile with potent activity in vitro and in vivo. PLoS One, 2022. 17(8): p. e0267859.
29. Pal, R., et al., Probiotics: insights and new opportunities for Clostridioides difficile intervention. Crit Rev Microbiol, 2022: p. 1-21.
30. Pal, R., M. Dai, and M. N. Seleem, High-throughput screening identifies a novel natural product-inspired scaffold capable of inhibiting Clostridioides difficile in vitro. Sci Rep, 2021. 11(1): p. 10913.
31. Abutaleb, N. S. and M. N. Seleem, In vivo efficacy of auranofin in a hamster model of Clostridioides difficile infection. Sci Rep, 2021. 11(1): p. 7093.
32. Naclerio, G. A., et al., Ultrapotent Inhibitor of Clostridioides difficile Growth, Which Suppresses Recurrence In Vivo. J Med Chem, 2020. 63(20): p. 11934-11944.
33. Abutaleb, N. S. and M. N. Seleem, Auranofin, at clinically achievable dose, protects mice andprevents recurrencefrom Clostridioides difficile infection. Sci Rep, 2020. 10(1): p. 7701.
34. St Cyr, S., et al., Update to CDC's Treatment Guidelines for Gonococcal Infection, 2020. MMWR Morb Mortal Wkly Rep, 2020. 69(50): p. 1911-1916.
35. Nuzzo, A., et al., Microbiome recovery in adultfemaleswith uncomplicatedurinary tract infections in a randomised phase 2A trial of the novel antibiotic gepotidacin (GSK140944). BMC Microbiol, 2021. 21(1): p. 181.
36. Acevedo, R., et al., The Global Meningococcal Initiative meeting on prevention of meningococcal disease worldwide: Epidemiology, surveillance, hypervirulent strains, antibiotic resistance and high-risk populations. Expert Rev Vaccines, 2019. 18(1): p. 15-30.
37. Bernet, E., et al., Sodium Tetraphenylborate Displays Selective Bactericidal Activity against Neisseria meningitidis and N. gonorrhoeae and Is Effective at Reducing Bacterial Infection Load. Antimicrob Agents Chemother, 2021. 65(2).
38. Vacca, P., et al., Neisseria meningitidis Antimicrobial Resistance in Italy, 2006 to 2016. Antimicrob Agents Chemother, 2018. 62(9).
39. Deghmane, A. E., E. Hong, and M. K. Taha, Emergence of meningococci with reduced susceptibility to third-generation cephalosporins. J Antimicrob Chemother, 2017. 72(1): p. 95-98.
40. Rouphael, N. G. and D. S. Stephens, Neisseria meningitidis: biology, microbiology, and epidemiology. Methods Mol Biol, 2012. 799: p. 1-20.
41. Smith, P. A., et al., Optimized arylomycins are a new class of Gram-negative antibiotics. Nature, 2018. 561(7722): p. 189-194.
42. Racine, E. and M. Gualtieri, From Worms to Drug Candidate: The Story of Odilorhabdins, a New Class of Antimicrobial Agents. Front Microbiol, 2019. 10: p. 2893.
43. Conly, J. and B. Johnston, Where are all the new antibiotics? The new antibiotic paradox. Can J Infect Dis Med Microbiol, 2005. 16(3): p. 159-60.
44. C. Girmenia, E. F., New-generation triazole antifungal drugs: review of the Phase II and III trials. Clinical Investigation, 2011. (11): p. 1577-1594.
45. Pasqualotto, A. C. and D. W. Denning, New and emerging treatments for fungal infections. J Antimicrob Chemother, 2008. 61 Suppl 1: p. i19-30.
46. Viljoen, J., et al., A phase 2, randomized, double-blind, multicenter trial to evaluate the safety and efficacy of three dosing regimens of isavuconazole compared with fluconazole in patients with uncomplicated esophageal candidiasis. Antimicrob Agents Chemother, 2015. 59(3): p. 1671-9.
47. Clemons, K. V. and D. A. Stevens, Efficacy of ravuconazole in treatment of mucosal candidosis in SCID mice. Antimicrob Agents Chemother, 2001. 45(12): p. 3433-6.
48. Read, P. J., et al., One confirmed and one suspected case of pharyngeal gonorrhoea treatment failure following 500 mg ceftriaxone in Sydney, Australia. Sex Health, 2013. 10(5): p. 460-2.
49. Unemo, M., et al., Treatment failure of pharyngeal gonorrhoea with internationally recommended first-line ceftriaxone verified in Slovenia, September 2011. Euro Surveill, 2012. 17(25).
50. Unemo, M., D. Golparian, and A. Hestner, Ceftriaxone treatment failure of pharyngeal gonorrhoea verified by international recommendations, Sweden, July 2010. Euro Surveill, 2011. 16(6).
51. Golparian, D., et al., Four treatment failures of pharyngeal gonorrhoea with ceftriaxone (500 mg) or cefotaxime (500 mg), Sweden, 2013 and 2014. Euro Surveill, 2014. 19(30).
52. Kong, F. Y. S. and J. S. Hocking, Treating pharyngeal gonorrhoea continues to remain a challenge. Lancet Infect Dis, 2022. 22(5): p. 573-574.
53. Regan, D. G., et al., Treatment for pharyngeal gonorrhoea under threat. Lancet Infect Dis, 2018. 18(11): p. 1175-1177.
54. M, Y. C., et al., Failure of 500 mg of ceftriaxone to eradicate pharyngeal gonorrhoea, Australia. J Antimicrob Chemother, 2013. 68(6): p. 1445-7.
55. Kinghorn, G., Pharyngeal gonorrhoea: a silent cause for concern. Sex Transm Infect, 2010. 86(6): p. 413-4.
56. Templeton, D. J., et al., Prevalence, incidence and risk factors for pharyngeal gonorrhoea in a community-based HIV-negative cohort of homosexual men in Sydney, Australia. Sex Transm Infect, 2010. 86(2): p. 90-6.
57. Tapsall, J., et al., Two cases of failed ceftriaxone treatment in pharyngeal gonorrhoea verified by molecular microbiological methods. J Med Microbiol, 2009. 58(Pt 5): p. 683-687.
58. Prevention, C.f.D.C.a., Vulvovaginal Candidiasis (VVC). Sexually Transmitted Infections Treatment Guidelines, 2021.
59. Chappell, B. T., et al., EVO100 prevents chlamydia and gonorrhea in women at high risk of infection. Am J Obstet Gynecol, 2021. 225(2): p. 162 el-162 e14.
60. Theuretzbacher, U., et al., Pharmacokinetic/pharmacodynamic considerations for new and current therapeutic drugs for uncomplicated gonorrhoea-challenges and opportunities. Clin Microbiol Infect, 2020. 26(12): p. 1630-1635.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A method of treating a Neisseria species (sp) infection or a symptom thereof in a subject in need thereof, the method comprising:
   administering, to the subject in need thereof, an amount of a triazole antifungal agent or a derivative thereof, or a prodrug thereof.
2. The method of aspect 1, wherein the Neisseria sp. is Neisseria gonorrhoeae or N. meningitidis.
3. The method of any one of aspects 1-2, wherein the triazole antifungal agent is ravuconazole, isavuconazole, itraconazole, ketoconazole, terconazole, or any combination thereof.
4. The method of aspect 3, wherein the amount of ravuconazole ranges from about 20 mg/kg to about 60 mg/kg.
5. The method of any one of aspects 3-4, wherein the amount of ravuconazole is about 40 mg/kg.
6. The method of any one of aspects 1-5, wherein the amount of the triazole antifungal agent ranges from about 10 mg to 1000 mg or more.
7. The method of any one of aspects 1-6, wherein the amount of the triazole antifungal agent is administered in a single dose.
8. The method of aspect 7, wherein the single dose is a single, one-time dose.
9. The method of any one of aspects 1-6, wherein the amount of the triazole antifungal agent is administered in two or more doses.
10. The method of any one of aspects 1-9, wherein the antifungal agent does not disrupt a microbiome in a subject in need thereof.
11. The method of any one of aspects 1-10, wherein administering an amount of a triazole antifungal agent comprises oral administration.
12. The method of any one of aspects 1-11, wherein administering an amount of a triazole antifungal agent comprises topical administration.
13. The method of any one of aspects 1-12, further comprising administering an amount of an antibiotic.
14. The method of aspect 13, wherein the antibiotic comprises azithromycin, penicillin, ciprofloxacin, ceftriaxone, cefixime, tetracycline, or any combination thereof.
15. The method of any one of aspects 12-13, wherein the amount of the antibiotic is about 0.001 μM to Ig or more.
16. The method of any one of aspects 2-15, wherein the Neisseria gonorrhoeae is an antibiotic resistant Neisseria gonorrhoeae.
17. The method of aspect 16, wherein the Neisseria gonorrhoeae is resistant to azithromycin, penicillin, ciprofloxacin, ceftriaxone, cefixime, tetracycline, or any combination thereof.
18. A pharmaceutical formulation comprising:
   an amount of a triazole antifungal agent or a derivative thereof, or a prodrug thereof effective to treat a Neisseria species (sp) infection in a subject in need thereof, and
   a pharmaceutically acceptable carrier.
19. The pharmaceutical formulation of aspect 18, wherein the Neisseria sp. Is Neisseria gonorrhoeae or N. meningitidis.
20. The pharmaceutical formulation of any one of aspects 18-19, wherein the triazole antifungal agent is ravuconazole, isavuconazole, itraconazole, ketoconazole, terconazole, or any combination thereof.
21. The pharmaceutical formulation of any one of aspects 18-20, wherein the amount of the amount of the triazole antifungal agent ranges from about 10 mg to 1000 mg or more.
22. The pharmaceutical formulation of any one of aspects 18-21, further comprising an amount of an antibiotic.
23. The pharmaceutical formulation of aspect 22, wherein the antibiotic is azithromycin, penicillin, ciprofloxacin, ceftriaxone, cefixime, tetracycline, or any combination thereof.
24. The pharmaceutical formulation of any one of aspects 22-23, wherein the amount of the antibiotic is about 0.001 μM to 1 g or more.
25. The pharmaceutical formulation of any one of aspects 18-24, wherein the pharmaceutical formulation is adapted for a single one time administration.
26. The pharmaceutical formulation of any one of aspects 18-25, wherein the pharmaceutical formulation is adapted for oral administration.
27. The pharmaceutical formulation of any one of aspects 18-25, wherein the pharmaceutical formulation is adapted for topical administration.
28. A kit comprising:
   a pharmaceutical formulation of any one of aspects 18-27, and instructions in a tangible medium of expression directing administration to a subject in need thereof, wherein the subject in need thereof has a Neisseria species (sp) infection.
29. The kit of aspect 28, wherein the Neisseria sp. is Neisseria gonorrhoeae or N. meningitidis.

1. A method of treating a Neisseria species (sp) infection or a symptom thereof in a subject in need thereof, the method comprising:
administering, to the subject in need thereof, an amount of a triazole antifungal agent or a derivative thereof, or a prodrug thereof.

2. The method of claim 1, wherein the Neisseria sp. is Neisseria gonorrhoeae or N. meningitidis.

3. The method of claim 1, wherein (a) the triazole antifungal agent is ravuconazole, isavuconazole, itraconazole, ketoconazole, terconazole, or any combination thereof, or (b) the amount of the amount of the triazole antifungal agent ranges from about 10 mg to 1000 mg or more, or (c) both (a) and (b).

4. The method of claim 3, wherein the amount of ravuconazole ranges from about 20 mg/kg to about 60 mg/kg or wherein the amount of ravuconazole is about 40 mg/kg.

5. (canceled)

6. (canceled)

7. The method of claim 1, wherein the amount of the triazole antifungal agent is administered in a single dose, in a single one-time dose, or in two or more doses.

8. (canceled)

9. (canceled)

10. The method of claim 1, wherein the antifungal agent does not disrupt a microbiome in a subject in need thereof.

11. The method of claim 1, wherein administering an amount of a triazole antifungal agent comprises oral administration or topical administration.

12. (canceled)

13. The method of claim 1, further comprising administering an amount of an antibiotic.

14. The method of claim 13, wherein (a) the antibiotic comprises azithromycin, penicillin, ciprofloxacin, ceftriaxone, cefixime, tetracycline, or any combination thereof, or (b) the amount of the antibiotic is about 0.001 μM to 1 g or more, or (c) both (a) and (b).

15. (canceled)

16. The method of claim 1, wherein the Neisseria gonorrhoeae is an antibiotic-resistant Neisseria gonorrhoeae.

17. The method of claim 16, wherein the Neisseria gonorrhoeae is resistant to azithromycin, penicillin, ciprofloxacin, ceftriaxone, cefixime, tetracycline, or any combination thereof.

18. A pharmaceutical formulation comprising:
an amount of a triazole antifungal agent or a derivative thereof, or a prodrug thereof effective to treat a Neisseria species (sp) infection in a subject in need thereof, and
a pharmaceutically acceptable carrier.

19. The pharmaceutical formulation of claim 18, wherein the Neisseria sp. Is Neisseria gonorrhoeae or N. meningitidis.

20. The pharmaceutical formulation of claim 18, wherein (a) the triazole antifungal agent is ravuconazole, isavuconazole, itraconazole, ketoconazole, terconazole, or any combination thereof, or (b) the amount of the amount of the triazole antifungal agent ranges from about 10 mg to 1000 mg or more, or (c) both (a) and (b).

21. (canceled)

22. The pharmaceutical formulation of claim 18, further comprising an amount of an antibiotic.

23. The pharmaceutical formulation of claim 22, wherein (a) the antibiotic is azithromycin, penicillin, ciprofloxacin, ceftriaxone, cefixime, tetracycline, or any combination thereof, or (b) the antibiotic is about 0.001 μM to 1 g or more, or (c) both (a) and (b).

24. (canceled)

25. The pharmaceutical formulation of claim 17, wherein the pharmaceutical formulation is adapted for a single one-time administration.

26. The pharmaceutical formulation of claim 18, wherein the pharmaceutical formulation is adapted for oral administration or is adapted for topical administration.

27. (canceled)

28. A kit comprising:
a pharmaceutical formulation of claim 18; and instructions in a tangible medium of expression directing administration to a subject in need thereof, wherein the subject in need thereof has a Neisseria species (sp) infection.

29. The kit of claim 28, wherein the Neisseria sp. is Neisseria gonorrhoeae or N. meningitidis.
